(19) United States
(12) Patent Application Publication
(10) Pub. No.: US 2024/0252122 A1
(43) Pub. Date: Aug. 1, 2024

(54) SLEEP APNEA-HYPOPNEA INDEX ESTIMATION APPARATUS, METHOD, AND COMPUTER-EXECUTABLE INSTRUCTIONS

(71) Applicant: HEALTH SENSING CO., LTD., Tokyo (JP)

(72) Inventors: Masatomo KANEGAE, Hachioji-city, Tokyo (JP); Kyuichi NIIZEKI, Hachioji-city, Tokyo (JP)

(21) Appl. No.: 18/289,664

(22) PCT Filed: Mar. 13, 2023
(86) PCT No.: PCT/JP2023/010494

§ 371 Date: Nov. 6, 2023

(30) Foreign Application Priority Data
Mar. 14, 2022 (JP) 2022-060807
Jun. 1, 2022 (JP) 2022-098448

(51) Int. Cl. A61B 5/00; A61B 5/08; A61B 5/11; A61B 5/113; A61B 90/00

(52) U.S. Cl. CPC A61B 5/7275; A61B 5/0816; A61B 5/1102; A61B 5/113; A61B 5/7257; A61B 5/726; A61B 5/7267; A61B 90/06; A61B 2090/064

(57) ABSTRACT

An apnea-hypopnea index is estimated without restraining a subject.

Bio-vibration signals are obtained from a subject during sleep in a non-contact and unrestrained manner. From the signals, 4 parameters of respiratory rate, heart rate, body movement, and phase coherence calculated from a difference in instantaneous phase between heartbeat interval variation and respiratory pattern are extracted and put into histograms, which are further transformed into a feature image. The feature image is input in an AHI estimation model that has undergone machine learning.

### TECHNICAL FIELD

The present invention relates to technologies for estimating sleep apnea-hypopnea index (AHI) from heart rate (HR), respiratory rate (RR), phase coherence (A) calculated from an instantaneous phase difference between heartbeat interval variation and respiratory pattern, and body movement rate (BM).

### BACKGROUND ART

A definitive diagnosis of sleep apnea syndrome (SAS) can be made by a gold-standard examination of polysomnography (PSG), but an apparatus for this examination itself is elaborate and measures too many bio-signals and thus is not suitable for SAS screening. When SAS is suspected, therefore, a simplified examination apparatus is generally used. In SAS screening, respiratory airflow and percutaneous arterial oxygen saturation (SpO₂) are measured with a pneumotachograph and a pulse oximeter, respectively, and an average number of “sleep apnea” and “hypopnea” episodes per hour is determined as an apnea-hypopnea index (AHI), which is included in the criteria for severity classification. Of note, sleep apnea is defined as an event in which respiratory airflow is disrupted for at least 10 seconds with a decrease in SpO₂ by 4% or more, and hypopnea is defined as an event of discernible reduction in airflow accompanied by either a decrease in SpO₂ by 3% to 4% or more or by signs of arousal.

Furthermore, a technology for estimating AHI in a non-contact and unrestrained manner has been proposed as a simplified screening method.

In an example of the technology disclosed, signals obtained with a piezoelectric sensor are subjected to band pass filter (BPF; 0.1-0.7 Hz) processing, noise-reduction processing, and Fourier transformation, and from a power of the low-frequency component of respiration, an apparent apnea-hypopnea index is calculated (Patent literature 1).

In another example of the technology disclosed, multiple continuous heartbeat intervals (R-R intervals or RRIs) output from a heart rate meter are input as feature vectors in a model that has undergone machine learning using a recurrent neural network (LSTM, etc.); whether sleep apnea or normal respiration can be determined only by the heartbeat intervals (Patent literature 2).

Both technologies are intended to simplify estimation of AHI using PSG. On the other hand, sleep apnea causes hypoxia, thereby puts the individual in a sympathetic dominant state of the autonomic nervous system, accompanied by a rapid rise in heart rate and an increased frequency of arousal during sleep, and potentially results in changes in body movements. Accordingly, a desirable method of indirect estimation of AHI includes comprehensive assessment of information containing data on autonomic nervous activities, which are affected by sleep apnea.

### PRECEDING TECHNICAL LITERATURE

### Patent Literature

[Patent literature 1] Jpn. unexamined patent publication 2018-011948
[Patent literature 2] WO2020/166239

### SUMMARY OF INVENTION

### Technical Problem

In view of such situation, the purpose of the present invention is to deliver a technology for estimating AHI from bio-vibration signals obtained from a subject in a non-contact and unrestrained manner. From the signals, 4 parameters of respiratory rate, heart rate, body movement, and phase coherence calculated from a difference in instantaneous phase between heartbeat interval variation and respiratory pattern are extracted and put into histograms, which are further transformed into a feature image. The feature image is input in an AHI estimation model that has undergone machine learning for the relationship between feature images and true AHI.

In addition, the purpose of the present invention is to deliver an alternative technology for estimating AHI from bio-vibration signals obtained from a subject in a non-contact and unrestrained manner. From the signals, 5 parameters of respiratory rate, heart rate, body movement, high-frequency component of heartbeat variation power spectrum, and a ratio of low-frequency to high-frequency component of heartbeat variation power spectrum are extracted and put into histograms, which are further transformed into feature images. The feature images are input in an AHI estimation model that has undergone machine learning for the relationship between feature images and true AHI.

### Solution to Problem

An apnea-hypopnea index estimation apparatus, comprising:
a bio-vibration signal receiving part configured to receive bio-vibration signals of an animal;
a body movement signal detection part configured to detect body movement signals from the bio-vibration signals;
a respiratory rate detection part configured to detect respiratory rate from the bio-vibration signals;
a heart rate detection part configured to detect heart rate from the bio-vibration signals;
a phase coherence computation part configured to calculate phase coherence from a difference in instantaneous phase between heartbeat interval variation and respiratory pattern detected from the bio-vibration signals;
a histogram creation part configured to receive the body movement signals, heart rate, respiratory rate, and phase coherence and create the respective histograms;
a feature image creation part configured to receive the respective histograms and create a feature image; and
an apnea-hypopnea index estimation part configured to receive the feature image and estimate an apnea-hypopnea index,
which undergoes machine learning using teaching data of AHI, which is clinically assessed by sleep polysomnography and input data of the feature image corresponding to the teaching data and then estimates AHI from the feature image of a subject entered.

Furthermore, a sensor part such as a sheet-type piezoelectric sensor is added to constitute an apnea-hypopnea index estimation system.

In place of phase coherence, high-frequency component and a ratio of low-frequency to high-frequency component of the heartbeat variation power spectrum are used.

### Advantageous Effect of Invention

The present invention is useful in screening of sleep apnea-hypopnea syndrome because AHI of a subject can be estimated without using a pneumotachograph or a pulse oximeter, which restrains the subject.

In addition, AHI is estimated from the feature image that includes multiple physiological indices over sleep time, and the image information enables objective screening of sleep apnea-hypopnea syndrome.

### BRIEF DESCRIPTION OF DRAWINGS

FIG. 1 Simplified block diagram of sleep apnea-hypopnea estimation apparatus

FIG. 2 Histograms of 4 physiological indices and the resultant feature image when AHI is 2

FIG. 3 Histograms of 4 physiological indices and the resultant feature image when AHI is 91

FIG. 4 Correlation of estimated AHI from the histograms of 4 physiological indices to true AHI

FIG. 5 Simplified block diagram of sleep apnea-hypopnea estimation apparatus

FIG. 6 Histograms of 5 physiological indices and the resultant feature image when AHI is 2

FIG. 7 Histograms of 5 physiological indices and the resultant feature image when AHI is 91

FIG. 8 Correlation of estimated AHI from the histograms of 5 physiological indices to true AHI

### DESCRIPTION OF EMBODIMENTS

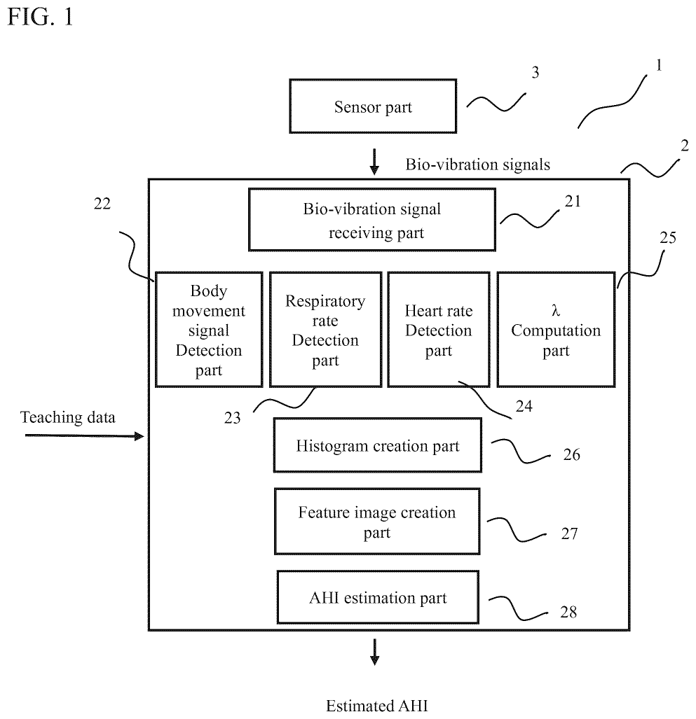

Using FIG. 1, an embodiment of the present invention is described. An AHI estimation system 1 is comprised of an AHI estimation apparatus 2 and a sensor part 3. Bio-vibration signals detected with the sensor part 3 or ones previously acquired and stored in a memory are input in the AHI estimation apparatus 2 to output estimated AHI.

The AHI estimation apparatus 2 is a core part of the present invention and comprised of a bio-vibration signal receiving part 21, a body movement signal detection part 22, a respiratory rate detection part 23, a heart rate detection part 24, a phase coherence (A) computation part 25, a histogram creation part 26, a feature image creation part 27, and an AHI estimation part 28.

Of note, each component is usually implemented in a computer such as a personal computer, a smartphone, or a tablet equipped with a CPU, a memory, an external memory, and communication means.

Each component is described in detail.

The sensor part 3 detects bio-vibrations of an animal and outputs bio-vibration signals. For example, the sensor part 3 is a sheet-type piezoelectric sensor, installed in a mat on which an animal lies, and outputs bio-vibration signals based on body movements, heartbeats (ballistocardiographic signals by heart beating), respiration, and vocalization, but the signals may include ones derived from vibrations caused by external environment, etc.

Of note, the sensor part 3 is not limited to the sheet-type piezoelectric sensor and may be, for example, a combination of a sphygmograph and an electrocardiograph.

The bio-vibration signal receiving part 21 receives bio-vibration signals output by the sensor part 3 through communication means such as cable or wireless.

The received bio-vibration signals are accumulated in the AHI estimation apparatus 2 and the external memory such as USB memory (not illustrated).

The body movement signal detection part 22 receives bio-vibration signals and outputs body movement signals. The bio-vibration signals include signals related to heartbeats and respiration, but the body movement signals have greater amplitude than ones related to heartbeats and respiration. The body movement signal detection part 22 converts the body movement signals into signals in appropriate amplitude and outputs them. In addition, the converted body movement signals are output as pulses and thus may be subjected to derivation processing, which allows counting of rises in a specified period and thereby outputting of the count of body movements in the specified period.

The respiratory rate detection part 23 receives bio-vibration signals and outputs respiratory rate. Because signals related to respiration included in the bio-vibration signals have much smaller amplitude than those related to body movement signals (usually smaller than 1/100 of that of the body movement signals), the following processing is required to calculate the respiratory rate.

The bio-vibration signals may be passed through a low pass filter (LPF) that allows signals at a frequency lower than 0.5 Hz to pass. Cutoff frequency of the LPF may be 0.3, 0.4, 0.6, 0.7, or 0.8 Hz.

Alternatively, in place of the low pass filer (LPF), a band pass filter (BPF) may be used. The lower limit of frequency for the BPF may be anything as long as it is adequately low. For example, 0.1 Hz may suffice.

The respiratory rate can be calculated by counting peaks of such obtained periodic respiration waveforms. Alternatively, bio-vibration signals may be subjected to Fourier or wavelet transformation to determine power spectrum, and peak frequency of the spectrum may be used to calculate respiratory rate.

The heart rate detection part 24 receives bio-vibration signals, extracts heartbeat signals, and calculate heart rate.

Because heartbeat signals included in the bio-vibration signals have much smaller amplitude than body movement signals (usually smaller than 1/100 of that of the body movement signals), the following processing is required to calculate the heart rate.
(1) The bio-vibration signals are processed with a BPF of 1 to 4 Hz, and periodic peaks in a specified period are counted. The band of the BPF is not limited to that from 1 to 4 Hz. The lower limit of frequency may be not less than 0.5, 0.6, 0.7, 0.8, 0.9, or 1 Hz, and the upper limit of frequency may be not more than 10, 8, 6, 5, or 3 Hz.
(2) The bio-vibration signals may be passed through a BPF with the lower limit of frequency not less than 5 Hz, and the filtered signals may be transformed into absolute values to extract the envelope or may be passed through an integration filter with a time constant around 0.1 seconds. The upper limit of frequency of the BPF may be not more than 10, 20, 30, or 40 Hz. The above integration filter processing may be replaced by quadrature detection processing, and from the processed waveforms, peak intervals are measured by time unit to determine heartbeat intervals.
(3) Bio-vibration signals in a specific period may be subjected to Fourier or wavelet transformation to determine power spectrum, and peak frequency of the spectrum may be used to calculate heart rate.

The phase coherence computation part 25 calculates phase coherence as an instantaneous phase difference between heartbeat interval variation and respiratory pattern.

The phase coherence computation part is comprised of a biological information acquisition means that acquires biological information on heartbeats, respiration, and others, a respiration waveform extraction means that extracts respiratory pattern, a heartbeat interval calculation means that calculates heartbeat interval variations, and a phase coherence calculation means that calculates phase coherence of an instantaneous phase difference between heartbeat interval variation and respiratory pattern. For details, see WO2017/141976 (an earlier application of the applicant)

The histogram creation part 26 prepares histograms of data on each of heart rate, respiratory rate, phase coherence, and body movement rate per certain period of time in specified sleep time. The specified sleep time represents, for example, one night.

The histogram of heart rate has a horizontal axis of minute heart rate rank (40-120) and a vertical axis of frequency.

The histogram of respiratory rate has a horizontal axis of minute respiratory rate rank (0-40) and a vertical axis of frequency.

The histogram of phase coherence has a horizontal axis of A rank (0-1) and a vertical axis of frequency.

The histogram of body movement has a horizontal axis of body movement proportion rank (0-0.5) and a vertical axis of frequency.

The body movement rate is determined as a moving average of body movement occurrences per 10 minutes by sequentially assessing bio-vibration signals collected in a 10-second window for presence or absence of body movement by the body movement signal detection part 22 while moving the window by 5 seconds after each assessment.

The feature image creation part 27 creates a feature image from these histograms. The width of the feature image corresponds to the rank of each histogram. For example, the width is divided into 50 segments (number of bins). The vertical components of the image represent heart rate (HR), respiratory rate (RR), phase coherence (A), and body movement rate (BM) in this order from the top. Accordingly, if each histogram has 50 bins, the image will have 200 picture elements (4×50=200).

Frequency at each rank in a histogram of heart rate (HR), respiratory rate (RR), phase coherence (λ), or body movement rate (BM) is represented by the corresponding image intensity.

This feature image is effective in objectively assessing AHI based on the image pattern because it represents a distribution of 4 parameters, heart rate, respiratory rate, λ, and body movement rate, in the bio-vibration signals collected from a subject in a certain sleep time. Of note, the number of bins is not limited to 50, and the image intensity correlated to frequency may be arbitrarily chosen as long as a change of frequency is represented by a change of the image intensity.

The AHI estimation part 28 is a deep learning apparatus comprised of an input layer with convolutional neural network (CNN) effective in image-based assessment and estimation, two fully connected layers at the middle layer, and a regression output layer.

In the deep learning apparatus with CNN, clinically assessed AHI based on PSG and the corresponding feature image are entered as teaching data and input data, respectively, for machine learning, and thereby an AHI estimation model (not illustrated) is constructed inside the AHI estimation part. The corresponding feature image is an image created based on histograms of heart rate (HR), respiratory rate (RR), phase coherence (λ), and body movement rate (BM) calculated from bio-vibration signals recorded at the same time with PSG, which are used in the estimation of clinically assessed AHI based on PSG.

The AHI estimation part 28 inputs the feature image of a subject in the AHI estimation model to estimate AHI. Estimated AHI is presented as the number of apnea-hypopnea episodes per hour.

The above explains each component of the AHI estimation system 1, but the present invention can function without the sensor part 3.

The AHI estimation apparatus 2 can receive bio-vibration signals previously acquired from a subject in a specified period through the communication means or USB memory, calculate heart rate (HR), respiratory rate (RR), phase coherence (λ), and body movement rate (BM), create their histograms, creates a feature image based on the histograms, input the feature image in the AHI estimation part 28 to estimate AHI of the subject.

In addition, accuracy of the AHI estimation may be improved using an AHI estimation model generated as follows: the heart rate detection part 24 calculates heart rate (HR) and its standard deviation (SDHR), the respiratory rate detection part 23 calculates respiratory rate (RR) and kurtosis of power spectrum of respiration signals (KRR), the histogram creation part 26 creates histograms of heart rate (HR), respiratory rate (RR), phase coherence (λ), and body movement rate (BM) as well as the standard deviation of HR (SDHR) and kurtosis of power spectrum of respiration signals (KRR), and the feature image creation part 27 creates feature images from these histograms; and using these feature images, the AHI estimation model is created.

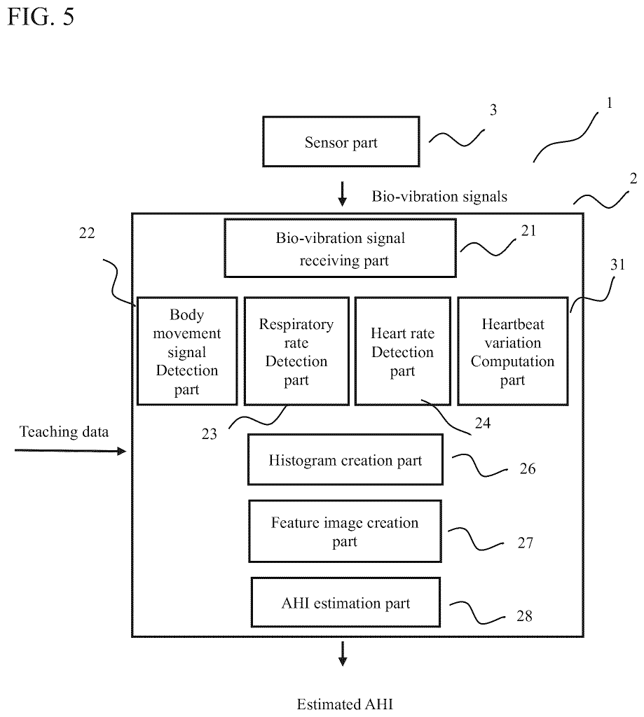

Using FIG. 5, another embodiment of the present invention is described.

The embodiment in FIG. 5 is equipped with a heartbeat variation computation part 31 in place of the phase coherence (λ) computation part 25 in FIG. 1. In this section, parts in which the embodiment in FIG. 5 differs from that in FIG. 1 are explained.

The heartbeat variation computation part 31 calculates high-frequency component (HF, 0.15-0.40 Hz), low-frequency component (LF, 0.04-0.15 Hz), and a ratio of low-frequency to high-frequency component (LF/HF) of the heartbeat variation power spectrum obtained by Fourier transformation of heart rate calculated from the bio-vibration signals by the heart rate detection part 24.

In addition, the histogram creation part 26 creates histograms of high-frequency component (HF) and a ratio of low-frequency to high-frequency component (LF/HF) of the heartbeat variation power spectrum in place of that of phase coherence (λ). Histograms of heart rate (HR), respiratory rate (RR), and body movement rate (BM) are created as done in the embodiment in FIG. 1.

The histogram of high-frequency component (HF) of the heartbeat variation power spectrum has a horizontal axis of HF rank (0-1) and a vertical axis of frequency.

The histogram of the ratio of low-frequency to high-frequency component (LF/HF) of the heartbeat variation power spectrum has a horizontal axis of LF/HF rank (0-10) and a vertical axis of frequency.

The feature image creation part 27 creates feature images using the histograms of high-frequency component (HF) and the ratio of low-frequency to high-frequency component (LF/HF) of the heartbeat variation power spectrum in place of that of phase coherence (λ). Histograms of heart rate (HR), respiratory rate (RR), and body movement rate (BM) are used as done in the embodiment in FIG. 1.

### Example 1

Firstly, data from 25 subjects were input in the learning apparatus with CNN of the apnea-hypopnea estimation apparatus in FIG. 1 and underwent deep learning. The training was performed using data from 24 subjects out of 25 subjects, leaving out 1 subject for leave-one-out cross-validation.

The teaching data are AHI based on PSG, while the input data are the feature image obtained from the bio-vibration signals measured simultaneously with PSG.

The feature image was created as follows.

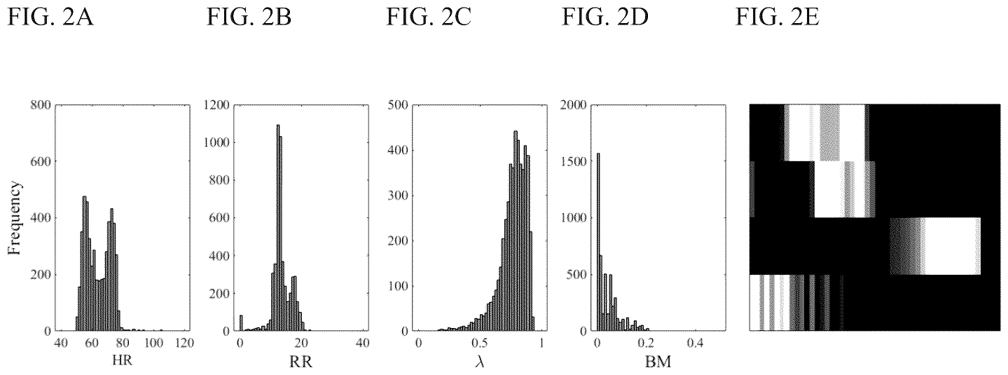

From the bio-vibration signals, heart rate (HR), respiratory rate (RR), phase coherence (λ), and body movement rate (BM) were calculated and then transformed into respective histograms. Next, one feature image in which the number of bins was expressed on the horizontal axis, and the above 4 parameters were set on the vertical axis was created by correlating frequency of each rank in the respective histograms to specific image intensity. FIG. 2 illustrates histograms and the resultant feature image from a subject with AHI of 2. Histograms of heart rate (HR), respiratory rate (RR), phase coherence (λ), and body movement rate (BM) are illustrated in this order from the left. The feature image created from these histograms is illustrated on the right.

In the feature image, the top column is derived from the histogram of HR, followed by the columns from those of RR, λ, and BM in this order, and frequency is represented by the corresponding image intensity.

The figures of a subject with AHI of 2 indicate that apnea-hypopnea was less frequent during sleep, and the histogram of heart rate (HR) presents two peaks indicative of deep sleep and REM sleep or arousal, suggesting that the heart rate was low during the deep sleep.

In addition, a tall and narrow distribution of the histogram of respiratory rate (RR) indicates less variations of respiratory rate.

The histogram of phase coherence (λ) presents a peak close to 1 and sparse data distribution below 0.5. The histogram of body movement rate (BM) presents a peak at zero (0), indicating that there were many periods of no body movement during sleep. The feature image reflects these features in the histograms of 4 parameters.

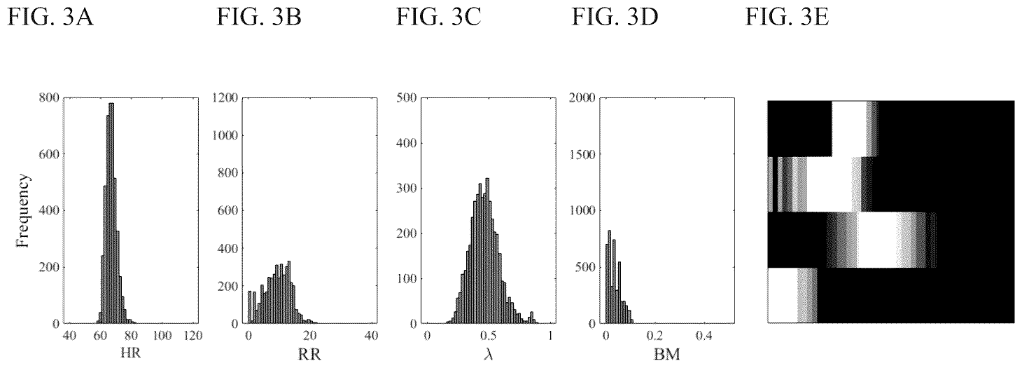

FIG. 3 illustrates histograms and the resultant feature image from a subject with AHI of 91. Histograms of heart rate (HR), respiratory rate (RR), phase coherence (λ), and body movement rate (BM) are illustrated in this order from the left. The feature image created from these histograms is illustrated on the right.

In the feature image, the top column is derived from the histogram of HR, followed by the columns from those of RR, λ, and BM in this order, and frequency is represented by the corresponding image intensity.

The figures of a subject with AHI of 91 indicate that frequency of apnea-hypopnea was high during sleep, and the histogram of heart rate (HR) does not present two peaks, each corresponding to a different sleep stage of deep sleep and REM sleep or arousal but presents the peak at higher heart rate than that from the subject with AHI of 2.

In addition, a peak in the histogram of respiratory rate (RR) is less sharp than that from the subject with AHI of 2, and the distribution spreads to a region of low respiratory rate, suggesting that apnea-hypopnea occurred and there were frequent variations in respiratory rate.

The histogram of phase coherence (λ) presents a peak near 0.5 and distribution to lower values than that from the subject with AHI of 2. The histogram of body movement rate (BM) presents a peak above zero (0), indicating that there were few periods of no body movement during sleep. The feature image reflects these features in the histograms of 4 parameters.

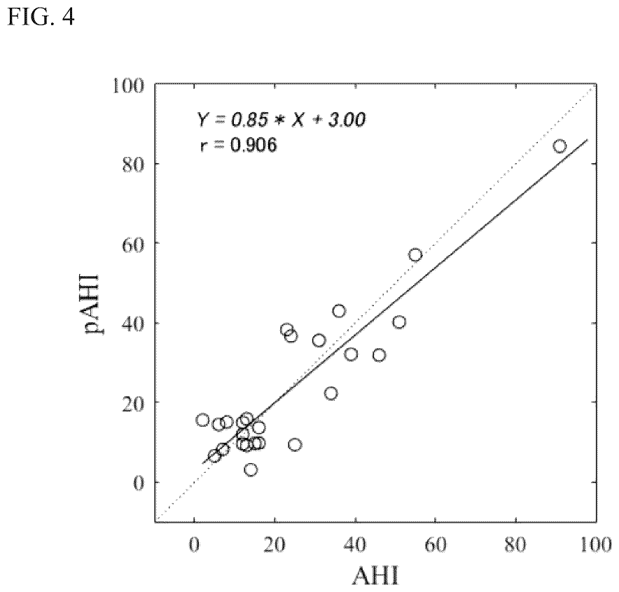

The feature image created as described above was input in the apnea-hypopnea estimation part to estimate AHI. FIG. 4 illustrates a relationship of estimated AHI (pAHI) to clinically assessed AHI, which was used as teaching signal. The good correlation was obtained with the correlation coefficient of 0.906.

As described above, respiratory rate, heart rate, phase coherence (λ), and body movement rate obtained from the bio-vibration signals during sleep include information related to sleep apnea-hypopnea syndrome, and input of the feature image of the aggregated information into the machine learning apparatus with CNN is confirmed to allow estimation of AHI.

### Example 2

An example of AHI estimation using high-frequency component (HF) and a ratio of low-frequency to high-frequency component (LF/HF) of the heartbeat variation power spectrum in place of that of phase coherence (λ) is explained below.

Firstly, data from 25 subjects were input in the learning apparatus with CNN of the apnea-hypopnea estimation apparatus in FIG. 5 and underwent deep learning. The training was performed using data from 24 subjects out of 25 subjects, leaving out 1 subject for leave-one-out cross-validation.

The teaching data are AHI based on PSG, while the input data are the feature image obtained from the bio-vibration signals measured simultaneously with PSG.

The feature image was created as follows.

From the bio-vibration signals, heart rate (HR), respiratory rate (RR), body movement rate (BM) as well as high-frequency component (HF) and a ratio of low-frequency to high-frequency component (LF/HF) of the heartbeat variation power spectrum were calculated and then transformed into respective histograms. Next, one feature image in which the number of bins was expressed on the horizontal axis, and the above 5 parameters were set on the vertical axis was created by correlating frequency of each rank in the respective histograms to specific image intensity.

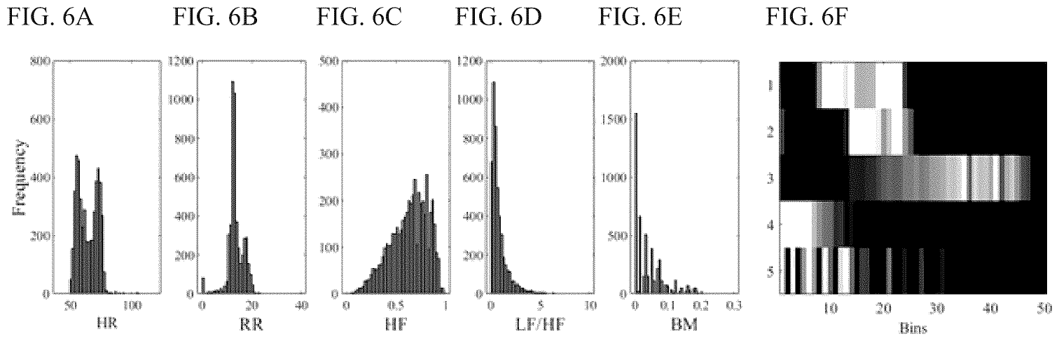

FIG. 6 illustrates histograms and the resultant feature image from a subject with AHI of 2. In this section, parts in which FIG. 6 differs from FIG. 2 are explained.

Histograms of heart rate (HR) (horizontal axis, 50 and 100; vertical axis, 0, 200, 400, 600, and 800), respiratory rate (RR) (horizontal axis, 0, 20, 40; vertical axis, 0, 200, 400, 600, 800, 1000, and 1200), high-frequency component (HF) (horizontal axis, 0, 0.5, and 1; vertical axis, 0, 100, 200, 300, 400, and 500), ratio of low-frequency to high-frequency component (LF/HF) (horizontal axis, 0, 5, and 10; vertical axis, 0, 200, 400, 600, 800, 1000, and 1200) of the heartbeat variation power spectrum, and body movement rate (BM) (horizontal axis, 0, 0.1, 0.2, and 0.3; vertical axis, 0, 500, 1000, 1500, and 2000) are illustrated in this order from the left.

The feature image created from these histograms is illustrated on the right. In the feature image, columns 1 to 5 are derived from the histograms of HR, RR, HF, LF/HF, and BM, respectively, and frequency is represented by the corresponding image intensity.

The histogram of HF presents a peak near 0.7, and that of LF/HF presents highly frequent appearance of low values.

The feature image reflects these features in the histograms of 5 parameters.

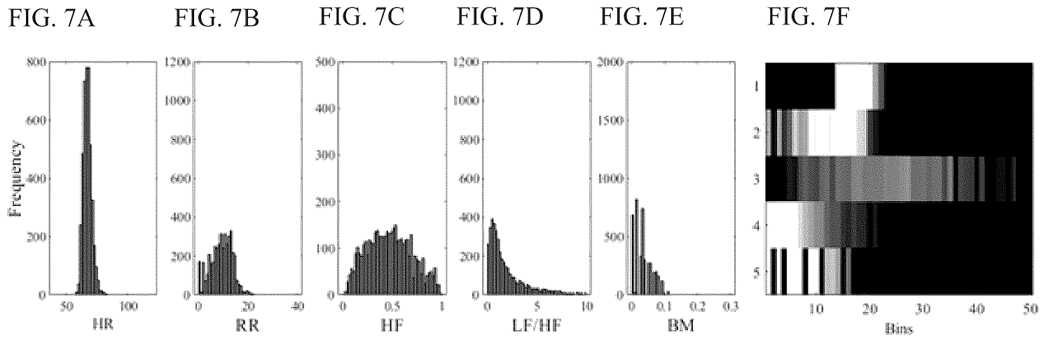

FIG. 7 illustrates histograms and the resultant feature image from a subject with AHI of 91. In this section, parts in which FIG. 7 differs from FIG. 3 are explained.

Histograms of heart rate (HR) (horizontal axis, 50 and 100; vertical axis, 0, 200, 400, 600, and 800), respiratory rate (RR) (horizontal axis, 0, 20, 40; vertical axis, 0, 200, 400, 600, 800, 1000, and 1200), high-frequency component (HF) (horizontal axis, 0, 0.5, and 1; vertical axis, 0, 100, 200, 300, 400, and 500), ratio of low-frequency to high-frequency component (LF/HF) (horizontal axis, 0, 5, and 10; vertical axis, 0, 200, 400, 600, 800, 1000, and 1200) of the heartbeat variation power spectrum, and body movement rate (BM) (horizontal axis, 0, 0.1, 0.2, and 0.3; vertical axis, 0, 500, 1000, 1500, and 2000) are illustrated in this order from the left.

The feature image created from these histograms is illustrated on the right.

In the feature image, columns 1 to 5 are derived from the histograms of HR, RR, HF, LF/HF, and BM, respectively, and frequency is represented by the corresponding image intensity.

The histogram of HF presents a peak at the median, and that of LF/HF presents less frequent appearances of low values and distribution to a region not less than 5 with increased frequency of appearance of high values.

The feature image reflects these features in the histograms of 5 parameters.

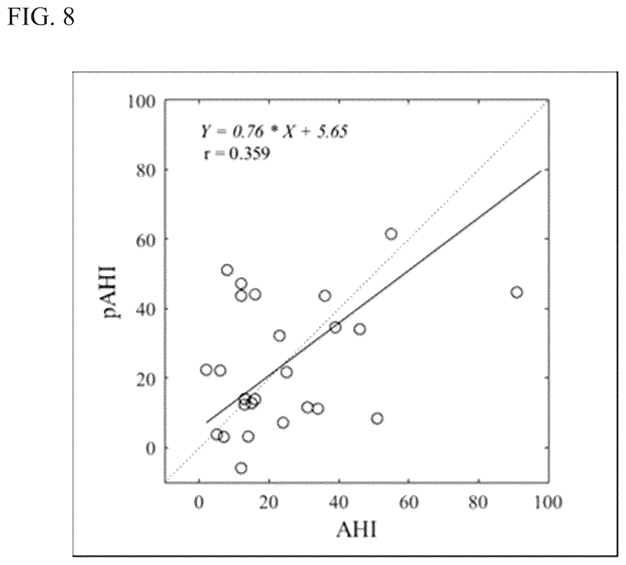

The feature image created as described above was input in the apnea-hypopnea estimation part to estimate AHI. FIG. 8 illustrates a relationship of estimated AHI (pAHI) to clinically assessed AHI, and the correlation coefficient of the fitted regression line was 0.359.

As described above, respiratory rate, heart rate, high-frequency component and the ratio of low-frequency to high-frequency component of the heartbeat variation power spectrum, and body movement rate obtained from the bio-vibration signals during sleep include information related to sleep apnea-hypopnea syndrome, and input of the feature image of the aggregated information into the machine learning apparatus with CNN is confirmed to allow estimation of AHI.

### REFERENCE SIGNS LIST

1 AHI estimation system
2 AHI estimation apparatus
21 Bio-vibration signal receiving part
22 Body movement signal detection part
23 Respiratory rate detection part
24 Heart rate detection part
25 Phase coherence (λ) computation part
26 Histogram creation part
27 Feature image creation part
28 AHI estimation part
3 Sensor part
31 Heartbeat variation computation part

1. An apnea-hypopnea index estimating apparatus comprising:
a bio-vibration signal receiving part configured to receive bio-vibration signals of an animal;
a body movement signal detection part configured to detect body movement signals from the bio-vibration signals;
a respiratory rate detection part configured to detect respiratory rate from the bio-vibration signals;
a heart rate detection part configured to detect heart rate from the bio-vibration signals;
a phase coherence computation part configured to calculate phase coherence from a difference in instantaneous phase between heartbeat interval variation and respiratory pattern detected from the bio-vibration signals;
a histogram creation part configured to receive the body movement signals, the heart rate, the respiratory rate, and h phase coherence and create histograms thereof;
a feature image creation part configured to receive the histograms and create a feature image from the histograms; and
an apnea-hypopnea index estimation part configured to perform machine learning with AHI clinically assessed based on sleep polysomnography, as teaching data, and the feature image corresponding to the teaching data, as input data, and estimate an apnea-hypopnea index in response to the feature image of a subject to be input.

2. The apnea-hypopnea index estimation apparatus according to claim 1, wherein the machine learning is performed through deep learning using a machine learning apparatus with CNN for the machine learning.

3. An apnea-hypopnea index estimation system, comprising:
the apnea-hypopnea index estimation apparatus according to claim 1; and
a sensor part configured to detect the bio-vibration signals of the animal.

4. A method for an apnea-hypopnea index estimation comprising the steps of:
receiving bio-vibration signals into a bio-vibration signal receiving part;
detecting body movement signals from the bio-vibration signals by a body movement signal detection part, detecting respiratory rate by a respiratory rate detection part, detecting heart rate by a heart rate detection part, and calculating phase coherence from a difference in instantaneous phase between heartbeat interval variation and respiratory pattern detected from the bio-vibration signals by a phase coherence computation part;
creating histograms of the body movement signals, the heart rate, the respiratory rate, and the phase coherence by a histogram creation part;
creating a feature image from the histograms by a feature image creation part;
performing machine learning with AHI clinically assessed based on sleep polysomnography, as a teaching data, and the feature image corresponding to the teaching data, as input data, to generate an apnea-hypopnea index estimation model by an apnea-hypopnea index estimation part; and
estimating an apnea-hypopnea index in response to the feature image of a subject to be input by an apnea-hypopnea index estimation part.

5. (canceled)

6. An apnea-hypopnea index estimating apparatus comprising:
a bio-vibration signal receiving part configured to receive bio-vibration signals of an animal;
a body movement signal detection part configured to detect body movement signals from the bio-vibration signals;
a respiratory rate detection part configured to detect respiratory rate from the bio-vibration signals;
a heart rate detection part configured to detect heart rate from the bio-vibration signals;
a heartbeat variation computation part configured to calculate high-frequency component and a ratio of low-frequency to high-frequency component of a heartbeat variation power spectrum from the heart rate;
a histogram creation part configured to receive the body movement signal, the heart rate, the respiratory rate, the high-frequency component of h heartbeat variation power spectrum, and h ratio of the low-frequency to the high-frequency component of h heartbeat variation power spectrum and create the histograms thereof;
a feature image creation part configured to receive the histograms and create a feature image from the histograms; and
an apnea-hypopnea index estimation part configured to perform machine learning with AHI clinically assessed based on sleep polysomnography, as teaching data, and the feature image corresponding to the teaching data, input data, and estimate an apnea-hypopnea index in response to the feature image of a subject to be input.

7. A non-transitory tangible computer-readable storage media, storing computer-executable instructions for apnea-hypopnea index estimation, the instructions, comprising:
receiving bio-vibration signals into a bio-vibration signal receiving part;
detecting body movement signals from the bio-vibration signals by a body movement signal detection part;
detecting respiratory rate from the bio-vibration signals by a respiratory rate detection part;
detecting heart rate from the bio-vibration signals by a heart rate detection part;
calculating phase coherence from a difference in instantaneous phase between heartbeat interval variation and respiratory pattern detected from the bio-vibration signals by a phase coherence computation part;
receiving the body movement signals, the heart rate, the respiratory rate, and the phase coherence and creating histograms thereof by a histogram creation part;
receiving the histograms and creating a feature image by a feature image creation part; and
performing machine learning with AHI clinically assessed based on sleep polysomnography, as teaching data, and the feature image corresponding to the teaching data, as input data by an apnea-hypopnea index estimation part; and
receiving the feature image of a subject and estimating an apnea-hypopnea index by an apnea-hypopnea index estimation part.
